(19) United States
(12) Patent Application Publication
(10) Pub. No.: US 2026/0139281 A1
(43) Pub. Date: May 21, 2026

(54) LOW ENERGY PROCESS FOR TRANSESTERIFICATION OF EXTRACTED LIPIDS FROM MICROBIAL DEGRADATION OF PLASTIC

(71) Applicant: Battelle Memorial Institute, Columbus, OH (US)

(72) Inventors: Daniel B. Garbark, Glen Dale, WV (US); Jeffrey T. Cafmeyer, Columbus, OH (US); Jacob Lilly, Dublin, OH (US)

(21) Appl. No.: 19/395,688

(22) Filed: Nov. 20, 2025

Related U.S. Application Data
(60) Provisional application No. 63/723,027, filed on Nov. 20, 2024.

(51) Int. Cl. C12P 7/6458; C12M 1/00; C12M 1/40; C12N 9/20

(52) U.S. Cl. CPC C12P 7/6458; C12M 21/18; C12M 29/08; C12N 9/20; C12Y 301/01003

(57) ABSTRACT

The invention provides a method and system for the enzymatic transesterification of fatty acids or fatty acid derived products to produce lubricant esters. The process involves a reaction mixture containing a fatty acid or fatty acid-derived substrate, an alcohol and a lipolytic enzyme catalyst. A reactive column with a porous frit is employed through which air is bubbled into a mixture of fatty acid derivatives, alcohol and an immobilized lipase catalyst. The air bubbles promote mixing and the subsequent stripping of volatile by-products, thereby increasing ester formation. This process can be carried out at ambient temperature, without the need for external heating or microwave irradiation. The method achieves greater that 99 mol % conversion to ester products at lower energy when compared to conventional thermochemical transesterification.

### GOVERNMENT RIGHTS

This invention was made with funding from the United States Government under contract number HR001120C0018, the government has certain rights in the invention.

### FIELD OF THE INVENTION

The present invention relates to green chemistry and biocatalytic processes, particularly to the low energy transesterification process for converting microbial lipids derived from plastic degradation into lubricant esters. This invention utilizes a reactive column system to facilitate the enzymatic esterification or transesterification under ambient temperature and low energy conditions.

### RELATED APPLICATIONS

This application claims the priority benefit of U.S. Provisional Patent Application Ser. No. 63/723,027 filed Nov. 20, 2024.

### INTRODUCTION

Fatty acids and fatty acid derivatives are essential precursors to manufacturing fuels, surfactants, lubricants and industrial intermediates. Current state of the art of converting triglycerides or fatty acid esters into lubricants typically rely on chemical catalysts such as sodium methoxide or metal alkoxides and require elevated temperatures (120-250° C.). These thermochemical methods are energy intensive and result in undesired side reactions, including epoxy ring opening, polymerization and oxidation or cross-linking of the fatty acid.

Recent advances have enabled microorganisms to degrade polymeric plastics and to metabolize them into lipids rich in fatty acid esters. These microbial oils represent a sustainable feedstock for lubricant synthesis. Due to their high viscosity, heterogeneity and low reactivity, thermochemical transesterification remains inefficient. For example, the current art of producing 2-ethylhexyl epoxy soyate, for use as plasticizer, reaches a maximum of ˜75% mol % conversion, which leaves behind 25% mol of unreacted epoxy methyl soyate. Attempts to enhance conversion through microwave heating requires temperatures in excess of 110° C. and high catalyst concentrations.

In view of these shortcomings, interest has increased for developing an energy efficient, environmentally benign method for converting microbial lipids into lubricant esters while maintaining a high conversion efficiency and product purity.

### SUMMARY OF THE INVENTION

The present disclosure is generally directed to the low energy, enzymatic conversion of fatty acids or fatty acid derived products by transesterification and the corresponding reactor system that enables the efficient conversion of microbial fatty acid derived products into lubricant esters at ambient temperature. Various embodiments contemplated herein may include, but need not be limited to one or more of the following:

In one aspect, the invention provides a method of esterifying a fatty acid or a fatty acid derived product comprising the steps of:
(a) providing a reaction mixture that comprises the fatty acid or fatty acid derived product, an alcohol, and a lipolytic enzyme;
(b) bubbling a gas through the reaction mixture to enhance agitation and removal of volatile by-products; and
wherein the alcohol has a boiling point at 1 atm of greater than 80° C., preferably greater than 90° C., and more preferably greater than 110° C.

In another aspect, the invention provides a reactor system for transesterification, comprising:
(a) a column;
(b) a porous frit or sparger disposed at the base of the column;
(c) a reaction mixture comprising a fatty acid or a fatty acid derived product, an alcohol, and a catalyst disposed over the frit; and
(d) a conduit adapted for feeding a gas through the frit to form gas bubbles to mix the catalyst and assist in stripping volatile components.

Any of the inventive aspects can be further characterized by one or any combination of the following: wherein the reaction is conducted at a temperature in the range of 15° C. to 30° C. or preferably between 20° C. to 25° C.; wherein the reaction is transesterification; wherein the fatty acid or fatty acid derived products are generated from microbial degradation of plastic; wherein the fatty acid or fatty acid derived product is esterified to a lubricant; wherein the lipolytic enzyme is a lipase immobilized on a solid resin and operate with air as the gas source; wherein the gas is atmospheric air; wherein the reaction is conducted in a reactor column; wherein the column contents are continuously stirred; wherein at least 99 mol % of the fatty acid or a fatty acid derived product is converted to an ester or esters; wherein the alcohol is a primary hydroxyl or secondary alcohol; wherein the alcohol is 2-butanol; wherein the alcohol is 2-ethylhexanol; wherein the alcohol is a branched alcohol; wherein the fatty acid or a fatty acid derived product is a fatty acid derived ester; wherein the reaction mixture comprises at least 2 wt % of each of at least three different fatty acids or fatty acid derived products; wherein the reaction mixture comprises a mixture of palmitic acid, stearic acid, and oleic acid or comprising a mixture of products derived from palmitic acid, stearic acid, and oleic acid; wherein air is passed through the frit from below the frit and leaves the column at the top; comprising culturing a microorganism under conditions sufficient to perform the step; wherein the microorganism may be naturally occurring or engineered; wherein the transesterification is performed following a first step of methanolysis or ethanolysis; wherein transesterification occurs at ambient temperature with continuous stirring in the presence of enzyme lipase; wherein the reaction is performed in a reactive column; wherein fatty acid or fatty acid derived product conversion to lubricants is >99% mole; wherein the transesterification product is an alcohol ester; wherein the transesterification product is 2-ethyl hexyl ester.

The invention may be further characterized by any selected descriptions from the data, for example, within ±30%, ±20% (or within ±10%) of any of the values in any of the data, tables or figures; however, the scope of the present invention, in its broader aspects, is not intended to be limited by these examples. As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of.” As used in this specification, the terms “includes” or “including” should not be read as limiting the invention but, rather, listing exemplary components. As is standard terminology, “systems” include to apparatus and materials (such as reactants and products) and conditions within the apparatus. All ranges are inclusive and combinable. For example, when a range of “1 to 5’ is recited, the recited range should be construed as including ranges “1 to 4”, “1 to 3”, “1-2”, “1-2 & 4-5”, “1-3 & 5”, “2-5”, any of 1, 2, 3, 4, or 5 individually, and the like.

### Glossary

The term “branched alcohol” refers to an alcohol in which at least one carbon atom in the hydrocarbon chain is substituted by one or more alkyl groups, yielding a non-linear molecular structure.

The term “bubble flow reactor” refers to a vessel in which gas is continuously bubbled through a liquid to enhance a reaction.

The term “conversion” or “mol % conversion” refers to the molar percentage of fatty acid reactant converted to corresponding ester product.

The term “engineered” is used herein, with reference to a microorganism to indicate that the microorganism contains at least one targeted genetic alteration introduced by man that distinguishes the engineered microorganism from the naturally occurring microorganism.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple and/or multiple names, sometimes depending on the source organism from which they derive. The enzyme names used herein encompass orthologs, including enzymes that may have one or more additional functions or a different name.

The term “extracted lipids” refers the fraction of microbial biomass soluble in organic solvents while comprising at least 50 wt % total fatty acid derivatives.

The term “fermentation” is used herein to refer to a process whereby a microbial cell converts one or more substrate(s) into a desired product (such as fatty acid) by means of one or more biological conversion steps, without the need for any chemical conversion step.

“Fatty acid” refers to monocarboxylic acids with aliphatic side chains that may be saturated or unsaturated. The term “fatty acid-derived product” is defined herein as a substrate comprising fatty acid alkyl esters, triglyceride, diglyceride, monoglyceride, free fatty acid or any combination thereof. Any oils and fats of vegetable or animal origin comprising fatty acids may be used as substrate for producing fatty acid alkyl esters in the process of the invention. Also fatty acid feedstock comprising fatty acid alkyl esters is suitable as feedstock for transesterification. The fatty acid feedstock maybe oil selected from the group consisting of: algae oil, canola oil, coconut oil, castor oil, corn oil, cottonseed oil, flax oil, fix oil, grape seed oil, hemp oil, jatropha oil, jojoba oil, mustard oil, palm oil, palm stearin, palm olein, palm kernel oil, peanut oil, rapeseed oil, rice bran oil, safflower oil, soybean oil, sunflower oil, tall oil and oil from halophytes, pennycress oil, camelina oil, jojoba oil, coriander oil, meadowfoam oil, seashore mallow oil, microbial oils or any combination thereof. The fatty acid may be fat selected from the group consisting of: animal fat, including tallow from pigs, beef and sheep, lard chicken fat, fish oil or any combination thereof. The fatty acid feedstock may be crude, refined, bleached, deodorized, degummed or any combination thereof.

The term “immobilized enzyme” refers to an enzyme bound to a solid support by covalent or physical adsorption, enabling easy separation and reuse.

“Lipases” (triacylglycerol acyl hydrolase; EC 3.1.1.3) belong to the esterase enzyme group and fats and oils are their natural carriers. Various microbes (yeasts, molds, bacteria) secrete lipases in their growth media, causing lipids to degrade. Lipases catalyze the hydrolysis reactions of oils and fats, but under suitable conditions they also catalyze the synthesis and transesterification of tri-, di-, and monoglyceride esters.

The term “low energy” refers to processes without external heat or microwave input.

A “lubricant” is an oil that functions to reduce friction between solid surfaces. The term “lubricant ester” as used herein, are esters prepared from mono-, di- or trialcohols and by transesterification.

The term “microbial fatty acids” or “microbe-derived fatty acids” refers to fatty acids and fatty acid derived products generated by microbial fermentation of plastic.

As used herein, the term “microorganism”, used interchangeably with “microbial cell”, refers to one of the following classes: bacteria, fungi, algae, eukaryote, archaea, protozoa and viruses. Suitable microorganisms refer to any of those well established and those novel microorganisms and variants that emerge from time to time.

The term “primary alcohol” contains a hydroxyl group bonded to a primary carbon atom. It is also defined as a molecule containing a “—CH₂OH” group.

The term “reaction mixture” refers to a liquid-phase composition comprising at least one fatty acid or derivative, at least one alcohol, and at least one catalyst with solvent. Here, “liquid-phase” includes slurry phase.

The term “secondary alcohol” has a formula “—CHROH” where “R” indicates a carbon-containing group.

The term “transesterification” refers to the process of exchanging the organic group of an ester with the organic group of an alcohol in the presence of a catalyst, which may be chemical, enzymatic, or biological.

### BRIEF DESCRIPTION OF DRAWINGS

The objects, features and advantages of the present invention will be more readily appreciated upon reference to the following disclosure when considered in conjunction with the accompanying drawings.

Table 1: Fatty acid methyl ester composition representative of the fatty acid methyl esters created from microbial decomposition of plastic.

Table 2: Viscosity and Pour Points from Traditional Transesterification.

Table 3: Viscosities and Pour Points of Lubricants from Various Equivalents of 2-Ethylhexanol.

FIG. 1: Reaction profiles for transesterification using three different alcohol catalysts as indicated.

FIG. 2: Representative laboratory scale column/bubble flow reactor.

FIG. 3: Lipase catalyzed transesterification with 2-ethylhexanol.

FIG. 4: Representative laboratory scale reactive column for transesterification to lubricant.

### DETAILED DESCRIPTION OF THE INVENTION

The invention provides a process for converting microbial fatty acid feedstocks into lubricant esters under mild conditions using an enzyme-catalyzed transesterification conducted in a gas sparged reactive column. Gas flow serves to remove volatile by-products to increase yield and to maintain uniform suspension of the immobilized enzyme catalyst.

The invention may provide an extended spectrum of activity in comparison to that obtained by each individual biological active component in the reactive column, and/or permit the use of lower amounts of the individual components when used in combination to that when used alone, in order to mediate effective activity.

The reactive column preferably comprises a glass or stainless steel cylindrical vessel containing a fritted plate. The enzyme catalyst can be introduced as immobilized particles dispersed within the liquid reaction mixture above the frit. Gas can be introduced through the frit at a controlled flow rate to introduce bubbles that gently mix the mixture and assist in removing the methanol byproduct.

The process of the invention is preferably performed in a “reactor column” (the invention can also be defined as a reaction system comprising the reaction vessel, reactants, optional solvents, and reaction conditions such as temperature) in which the catalyst may be freely distributed in the reaction mixture. The process applies an immobilized enzyme composition. A gas flow serves to remove volatile reaction products, for example methanol in a transesterification reaction, and hence to shift the equilibrium to the product side. The gas flow further serves to mix the reaction mixture. In a preferred embodiment wherein the immobilized enzyme is used, the gas flow serves to keep the catalyst suspended and in contact with the reaction mixture.

During the reaction, the temperature of the reaction mixture is preferably kept between 10° C. and 60° C., more preferably between 15° C. and 40° C., and more preferably between 15° C. and 30° C. The branched alcohol may comprise one or more alcohols or a mixture of two or more alcohols. The reaction column suitable for comprising the reaction mixture comprises at least one gas frit for the flow of gas through the column. The reactor system preferably comprises one or more gas pumps capable of delivering a gas flow, which can be passed through the reactor column. The gas pump may be any device suitable for creating a gas flow.

### Example 1: Transesterification with Branched Alcohols

A goal of the invention is conversion of fatty acid esters to lubricant esters by transesterification with branched alcohols. Branched alcohols inhibit crystallization yielding lower pour points, enhanced hydrolytic stability, and leading to variations in viscosity. This is done by creating molecular branching or “kinks” in the chains that lead to uneven molecular packing and protecting ester groups from hydrolysis. Utilizing past experience, we focused on transesterification with three alcohols; trimethylolpropane (TMP), 2-ethylhexanol, and 4-methyl-2-pentanol. For process evaluation, a stock solution of fatty acid methyl esters was synthesized to be representative of the fatty acid methyl esters created from microbial decomposition of plastic. The fatty acid methyl ester composition can be seen in Table 1.

TABLE 1
Fatty Acid Distribution

| Acid | C:Olefin | Wt % |
|---|---|---|
| Lauric | 12:0 | 0.00 |
| Myristic | 14:0 | 3.33 |
| Palmitic | 16:0 | 30.60 |
| Palmitoleic | 16:1 | 0.00 |
| Margaric | 17:0 | 0.00 |
| Cis-10-Heptanoic | 17:1 | 0.00 |
| Stearic | 18:0 | 7.85 |
| Oleic | 18:1 | 53.43 |
| Linoleic | 18:2 | 2.87 |
| Linolenic | 18:3 | 1.31 |
| Arachdic | 20:0 | 0.60 |
| Gadoleic | 20:1 | 0.00 |
| Behenic | 22:0 | 0.00 |
| Brassidic | 22:1 | 0.00 |
| | Total | 100.00 |

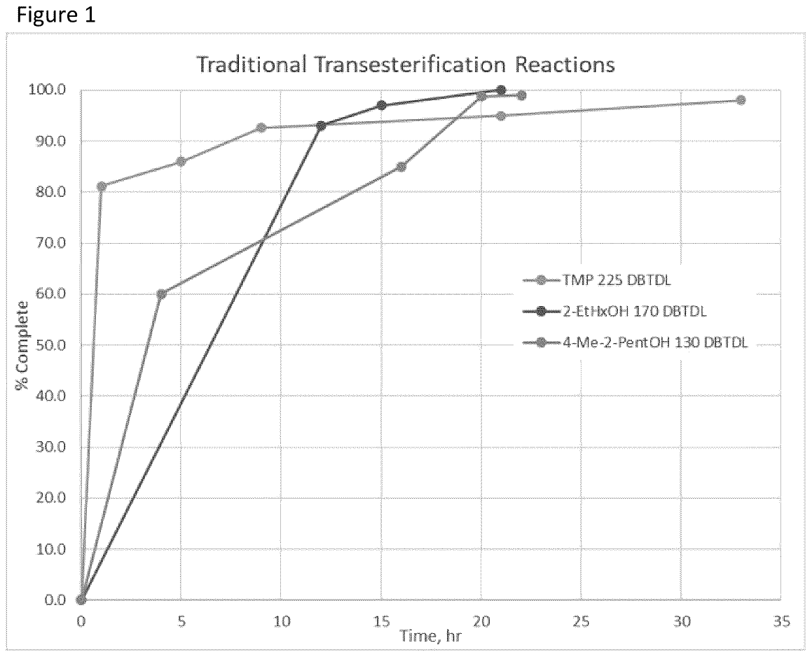

Traditional transesterification was initially used to determine baseline reaction characteristics and lubricant properties with the three alcohols. Dibutyltin dilaurate (DBTDL) was used as transesterification catalyst. The baseline reactions were run in round bottomed flasks with magnetic stirring, internal thermal couple, heating mantel, and a short path distillation apparatus. Temperature varied depending on the boiling point of the reactant alcohol. As trimethylolpropane had the highest boiling point, reaction temperature was raised to 225° C., and percent completion was followed over time. The TMP quickly reacts to form the diester as it has all primary hydroxyls. However, the formation of the triester took significantly longer to complete likely due to stearic hindrance. Overall, the reaction took 33 hours to reach 98.0% mol completion. The next highest boiling point reactant alcohol was 2-ethylhexanol which contained a primary hydroxyl making it highly reactive and quickly forming a 2-ethylhexyl monoester. The monoester versus triester was important as it reduced viscosity. Viscosity can be strongly correlated to lubricant molecular weight. The 2-ethylhexanol reaction was performed at 170° C. and reached 100% mol conversion after 21 hours. The third alcohol evaluated was 4-methyl-2-pentanol which was expected to react slower due to the presence of the secondary alcohol. The reaction was run at 130° C. for 22 hours reaching a conversion of 99.0% mol. The graph of the 3 reaction profiles can be seen in FIG. 1.

All three reactions required significant energy based on temperature and time while achieving greater than or equal to 98% mol conversion. While the TMP reaction required no purification, the other two alcohols required excess alcohol to be removed by heat and vacuum. These new base oils were evaluated for simulated pour point and viscosity. As expected, the TMP base oil contained the highest viscosity at 47 cSt at 40° C. Typically, greater branching gives lower pour points. However, the TMP ester product gave the second highest pour point. Longer chain saturates (C14 and larger) are detrimental to pour points due to greater crystallization potentials. The TMP lubricant sample contained a large percent weight of the fatty acids yielding a poor pour point. The effect of percent weight saturates could also be seen in the pour point of the 4-methyl-2-pentanol. While the viscosity of the 4-methyl-2-pentyl ester was expected to give the lowest viscosity based on molecular weight, the pour point was expected to be improved based on chain branching. Under these reaction conditions and with this fatty acid mixture, the branching was not able to overcome the crystallization leading to a higher pour point. The table of viscosity and pour points can be seen in Table 2.

TABLE 2
Traditional Transesterification Method

| Alcohol | 40° C. viscosity, cSt | Pour Point, ° C. |
|---|---|---|
| TMP | 47 | −6 |
| 2-Ethylhexanol | 12 | −24 |
| 4-Methyl-2-Pentanol | 6 | 0 |

### Example 2: Enzymatic Transesterification

To reduce energy use, we next began evaluation of enzymatic transesterification. The enzyme chosen for evaluation was Novozyme® 435 purchased from Strem Catalog number 06-3123 (resin supported Lipase). Initially, reactions were run in the same round bottomed reactors as were used for the production traditional transesterification samples. The reaction was performed at 60° C. 2-ethylhexanol reached a conversion of 100.0% mol in 9 hours. The 4-methyl-2-pentanol reaction reached a conversion of 99.9% mol in 12 hours. The TMP base oil was produced at 60° C. and 20 mm Hg vacuum as the reactivity was expected to be slow. A conversion of 98.2% mol was attained but required 143 hours. The reaction was significantly slower because of hindrance and mixture viscosity inhibiting the lipase catalyzed reaction.

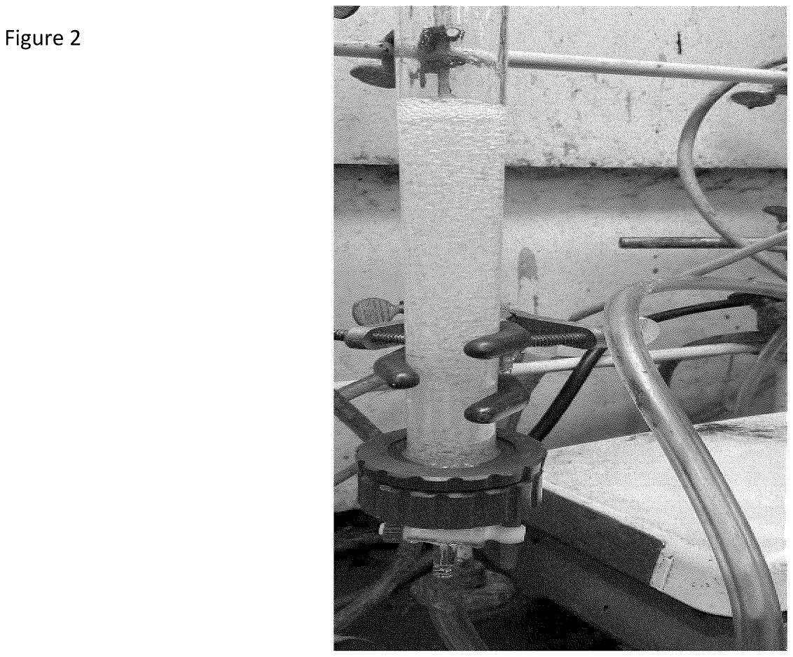

Because the TMP under both processes required significant time and energy, and 2-ethylhexanol had fast conversion, focus changed to improving the production of 2-ethylhexyl lubricant. A reactive column system was developed to run at ambient temperature using air floatation to both mix the resin supported lipase while aiding in the removal of methanol driving the transesterification forward. This reactor contained a glass fritted filter allowing for easy filtration of the resin after reaction completion. This reactor system can be easily scaled and controlled for in field operation. A picture of the reactor can be seen in FIG. 2.

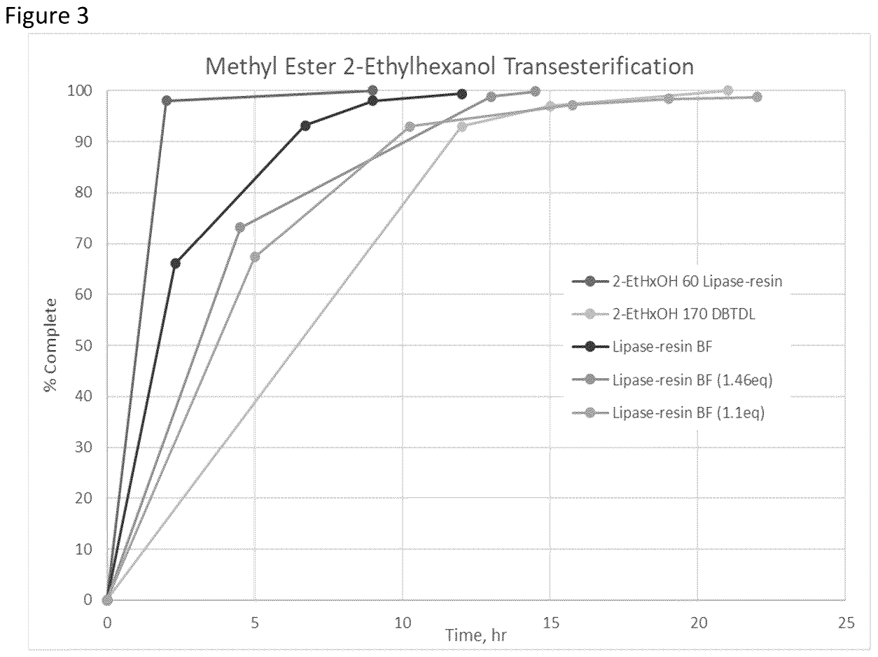

We initially evaluated a 5 equivalent excess of 2-ethylhexanol in the reactive column at 20° C. using a 0.5 standard cubic feet per hour (SCFH) flow of air. The reaction reached a conversion of 99.4% mol after 12 hours. Using this level of excess alcohol requires purification by vacuum distillation of the unreacted alcohol leading to more energy use and field impracticality. Because of this, and the relatively fast reaction time, we evaluated two other 2-ethylhexanol equivalent levels of 1.46 and 1.1. The results can be seen in FIG. 3. Here, all three equivalent levels achieved a conversion of approximately 99% mol in reasonable times (<24 hours). The BF designation in the figure stands for bubble flow meaning the reactor type used. Reaction time can be adjusted to yield the most benefit without using excessive amounts of excess alcohol. When excess alcohol is not purified, the lubricant has improved pour points through dilution as 2-ethylhexanol has a melting point of −76° C. The viscosity and pour point results for the various equivalents can be seen in Table 3 below. The viscosity and pour points remain relatively the same across the reaction spectrum. The 5 equivalent sample had excess alcohol removed by vacuum distillation.

TABLE 3
Lipase Transesterification Method

| Alcohol | 40° C. viscosity, cSt | Pour Point, ° C. |
|---|---|---|
| 2-Ethylhexanol (5 eq) | 9 | −31.8 |
| 2-Ethylhexanol (1.46 eq) | 7 | −30.0 |
| 2-Ethylhexanol (1.1 eq) | 8 | −29.5 |

### Example 3: Transesterification without Methanolysis

It was necessary to determine if the same enzyme could transesterify the crude extract when methanolysis was not performed prior. Initially, we used a standard triglyceride (soybean oil) and 1.1 equivalents of 2-ethylhexanol to verify conversions. After 20 hours of reaction time at ambient temperature, the mixture contained 83.5% mol ester product and 8.7% mol triglyceride. The reaction was stirred for 71 hours longer (91 hours total) and contained 86.0% mol ester product with 3.0% triglyceride remaining. While this reaction did not run to complete conversion, it showed that the enzyme is effective in transesterification. If greater equivalents of 2-ethylhexanol were used, the conversion would undoubtedly be higher. We next applied this process to the extracted lipids produced from extraction of the microbial cells with ethyl acetate instead of performing methanolysis prior to extraction. The reaction involved 1.1 theoretical equivalents of 2-ethylhexanol and resin supported lipase at ambient temperature. After 20 hours of reaction time, the mixture was fully transesterified to the desired 2-ethylhexyl ester product.

### Example 4: Transesterification in a Reactive Column

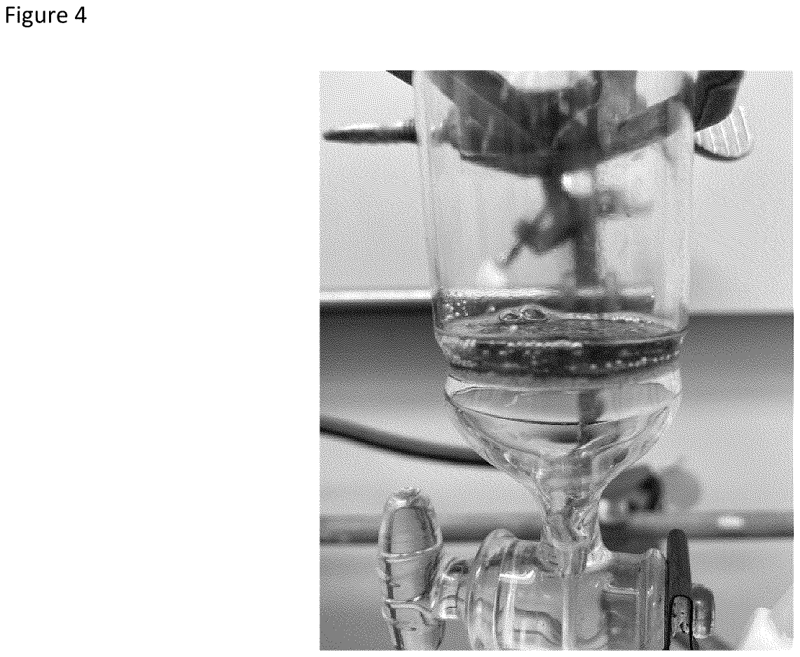

The reactive column was used for the final demonstration of transesterification. As mentioned earlier, the sparged column reactor supplies good mixing while the enzyme performs well at ambient temperature. Because of the small-scale amount of intermediate available (extracted lipids), the column was scaled down. A picture of the reaction can be seen in FIG. 4. Air was bubbled through the reactor at about 4 Ipm and allowed to run for two days. Successful transesterification was verified by NMR spectroscopy. For reference, to convert 1.5 grams of extracted lipids, you need a minimum of 0.75 grams of 2-ethylhexanol and 0.08 grams of resin supported lipase. To speed the reaction without requiring purification and decrease viscosity, 1.02 grams (1.5 equivalents) of 2-ethylhexanol are more ideal.

### Example 5: Transesterification of Epoxymethyl Soyate

Here, transesterification of epoxizided fatty acid esters is performed in a bubble flow reactor. The reactor set-up uses air flow from bottom through a frit to the top of the reactive column. The air flow serves two purposes. First it carries the methanol created through transesterification out of the reactor allowing for faster and efficient transesterification. The second purpose is to mix the resin throughout the column allowing for greater interaction of the enzyme with the reactor components. 30.86 g of epoxidized methyl high oleic soyate is dissolved in 40.12 g of 2-butanol and added to a fritted column. 2.67 g of lipase (resin supported novozyme 435) was then added and the reactor bubbled with air at 0.6SCFH. After 43.5 h, the liquid was filtered through the glass frit to remove resin and found to be 99.4% mol 2-butyl ester. Reactivity can be enhanced by using a less hindered primary hydroxyl alcohol such as 2-ethylhexanol. This product was chosen as a precursor to a low pour point soy-based lubricant with increased hydrolytic stability.

1. A method of esterifying a fatty acid or a fatty acid derived product comprising the steps of:
(a) providing a reaction mixture that comprises the fatty acid or fatty acid derived product, an alcohol, and a lipolytic enzyme;
(b) bubbling a gas through the reaction mixture;
wherein the alcohol has a boiling point at 1 atm of greater than 80° C.

2. The method of claim 1 wherein the reaction is conducted at a temperature in the range of 15° C. to 30° C. or 20° C. to 25° C.

3. The method of claim 1 wherein the reaction is transesterification.

4. The method of claim 1, wherein the fatty acid or fatty acid derived products are generated from microbial degradation of plastic.

5. The method of claim 1 wherein the fatty acid or fatty acid derived product is esterified to a lubricant.

6. The method of claim 1 wherein the lipolytic enzyme is a lipase.

7. The method of claim 1 wherein the gas is atmospheric air.

8. The method of claim 1 conducted in a reactor column with a porous frit.

9. The method of claim 8 wherein the column contents are continuously stirred by the gas flow.

10. The method of claim 1 wherein at least 99 mol % of the fatty acid or a fatty acid derived product is converted to an ester or esters.

11. The method of claim 1 wherein the alcohol is a primary or secondary alcohol.

12. The method of claim 11 wherein the alcohol is 2-butanol.

13. The method of claim 11 wherein the alcohol is 2-ethylhexanol.

14. The method of claim 11 wherein the alcohol is a branched alcohol.

15. The method of claim 1 wherein the fatty acid or a fatty acid derived product is an ester.

16. The method of claim 1 wherein the reaction mixture comprises at least 2 wt % of each of at least three different fatty acids or fatty acid derived products.

17. The method of claim 1 wherein the reaction mixture comprises a mixture of palmitic acid, stearic acid, and oleic acid or comprising a mixture of products derived from palmitic acid, stearic acid, and oleic acid.

18. The method of claim 1 wherein the lipolytic enzyme is immobilized on a solid matrix.

19.-20. (canceled)

21. A reactor system for transesterification, comprising:
(a) a column;
(b) a porous frit disposed in the column;
(c) a reaction mixture comprising a fatty acid or a fatty acid derived product, an alcohol, and a catalyst disposed over the frit; and
(d) a conduit adapted for feeding a gas through the frit to form gas bubbles in the reaction mixture.

22. The reactor column in claim 1 wherein air is passed through the frit from below the frit and leaves the column at the top.
